(19) United States
(12) Patent Application Publication
(10) Pub. No.: US 2019/0254369 A1
(43) Pub. Date: Aug. 22, 2019

(54) Underwear for Incontinence, Urological & Intestinal Conditions

(71) Applicants: Maria Teresa Crespo, Coral Gables, FL (US); Isabella Duarte-Crespo, Coral Gables, FL (US)

(72) Inventors: Maria Teresa Crespo, Coral Gables, FL (US); Isabella Duarte-Crespo, Coral Gables, FL (US)

(21) Appl. No.: 15/898,681

(22) Filed: Feb. 19, 2018

(51) Int. Cl. A41D 27/20; A41B 9/00; A61F 15/00; A61M 25/00

(52) U.S. Cl. CPC A41D 27/208; A41B 9/001; A41D 2300/50; A61F 15/001; A61M 25/002; A41D 27/201

(57) ABSTRACT

The invention relates to a novel underwear with two secured and hidden pockets placed below the waist band of the front area of the garment below the navel. An underwear with one non-detachable secured discreet pocket adapted to hold medical and personal supplies such as, portable catheters, wipes, pads/liners, and the other pocket being a secured yet detachable water resistant pocket adapted to hold wipes, pads/liners and disposable underwear.

### TECHNICAL FIELD

The present invention relates generally to the field of underwear with secured, concealed, detachable and water resistant pockets, of which the following is a specification, reference also being had to the accompanying drawing, forming a part hereof.

### CROSS-REFERENCE TO RELATED APPLICATIONS

None.

### BACKGROUND AND FIELD OF THE INVENTION

The invention most closely corresponds with USPTO US Class A41B9/002, relating to underpants or briefs, and sub-class A41D27/20, relating to pockets; making or setting-in pockets. The invention relates to an underwear with secured, concealed, detachable and water resistant pockets, wherein the invention is comprised of an interchangeable underwear, which can be modified from underwear to bag and bag to underwear, and contains two internal pockets, one being a secured, undetectable and detachable small bag/pocket made of water resistant material and hidden on the inner part of the underwear, and the other just an concealed secured pocket. This type of underwear can be utilized for and amongst other things by people suffering from either urological (i.e. incontinence, MS, etc.) or intestinal conditions (i.e. crohn disease, PTN) to store their medications, medical supplies, clean replacement underwear and soiled underwear.

Notwithstanding the aforementioned, it should be noted that the invention is not limited in form to the number of different materials that can be utilized to create the invention and/or articles of fabric clothing that can be used simultaneously at any one time and/or the shape of human clothing and/or pockets, as they could take just about any other shape imaginable.

### DESCRIPTION AND CRITICISM OF PRIOR ART

The invention improves upon the prior art, including those referenced in the relevant prior art section noted below. An issue with current underwear which contain pockets is that, they do not contain effectively concealed and secured pockets, nor do they contain detachable pockets which are made of water resistant material and which allow for an easy and effective means of storing soiled underwear and/or for privately and securely concealing the storage of necessary medical supplies, while also providing for the storage of a clean replacement underwear.

There is no prior art which is an interchangeable underwear, which can be modified from underwear to bag and bag to underwear, and which contains two internal pockets, one being a secured, undetectable and detachable small bag/pocket made of water resistant material and hidden on the inner part of the underwear, and the other just an concealed secured pocket, which can be utilized for and amongst other things by people suffering from either urological (i.e. incontinence, MS, etc.) or intestinal conditions (i.e. crohn disease, PTN) to store their medications, medical supplies, clean replacement underwear and soiled underwear.

Notwithstanding the aforementioned, it should be noted that the invention is not limited in form to the number of different shapes and/or articles of clothing or pockets that can be used, as it could take just about any other shape imaginable.

RELEVANT PRIOR ART INCLUDES

| Publication Number | Pub. Date | Assignee |
|---|---|---|
| U.S. PUB#. 20120311758A1 | 2012 Dec. 13, | Dollface Designs, LLC. |
| U.S. PUB#. 20140259306A1 | 2014 Sep. 18, | Dreamkassh Entertain- |
| | | ment LLC. |
| U.S. PUB#. 20150359273A1 | 2015 Dec. 17, | Gregg Olson. |
| U.S. PUB#. D775782S1 | 2017 Jan. 10, | Deborah Hall Peete. |
| U.S. PUB#. 6772446B1 | 2004 Aug. 10, | Ronnell Black. |
| U.S. PUB#. 6308340B1 | 2001 Oct. 30, | Rhonda Cook. |
| U.S. PUB#. 6018823A | 2000 Feb. 1, | Ortmeier; Suzan M. |
| U.S. PUB#. 3137862A | 1964 Jun. 23, | Fleet Post Office. |

### THE INVENTION

### Summary, Objects, Advantages and References

The following is a short summary and does not intend to limit the scope of the invention.

Embodiments described herein relate to and comprised an interchangeable underwear, which can be modified from underwear to bag and bag to underwear, and contains two internal pockets, one being a secured, undetectable and detachable small bag/pocket made of water resistant material and hidden on the inner part of the underwear, and the other just an concealed secured pocket. This type of underwear can be utilized for and amongst other things by people suffering from either urological (i.e. incontinence, MS, etc.) or intestinal conditions (i.e. crohn disease, PTN) to store their medications, medical supplies, clean replacement underwear and soiled underwear.

Finally, The fact that none of the prior art, representing numerous undergarments with pockets or similar orifices and other related prior art, have thus far created a product like the invention, proves that the invention is new functional improvement and non-obvious. Therefore, the invention is a new, unique, holistic, sustainable, and non-invasive remedy for and amongst other things by people suffering from either urological (i.e. incontinence, MS, etc.) or intestinal conditions (i.e. crohn disease, PTN) to store their medications, medical supplies, clean replacement underwear and soiled underwear, which no prior art has achieved thus far.

### BRIEF DESCRIPTION OF THE DRAWINGS

The drawing accompanying and forming part of this specification is included to depict certain aspects of the invention. A clearer impression of the invention, and of the components and operation of systems provided with the invention, will become more readily apparent by referring to the exemplary, and therefore non-limiting, embodiments illustrated in the drawing. Note that the features illustrated in the drawing are not necessarily drawn to scale.

FIG. 1—is an environmental perspective view illustrating the invention as a underwear, and showing its general features 1, 2, and 3 therein, which include secured, concealed, detachable and water resistant pockets, showing that the invention may be constructed with any outer surface of any article of clothing.

### DRAWINGS-REFERENCE NUMERALS

1—the invention

2—secured, concealed and non-detachable pocket

3—secured, concealed, detachable and water resistant pocket

### DETAILED DESCRIPTION, INCLUDING BEST MODES OF CARRYING OUT THE INVENTION

The following detailed description illustrates the invention by way of example, not by way of limitation of the principles of the invention. This description will clearly enable one skilled in the art to make and use the invention, and describes various embodiments, adaptations, variations, alternatives, and uses of the invention. The description and specification includes what are presently believed to be the best modes of carrying out the invention.

Herein, the terms “comprises,” “comprising,” “includes,” “including,” “has,” “having,” or any other variation, are intended to cover a non-exclusive inclusion. For example, a method, article, or apparatus that comprises a list of elements is not necessarily limited to only those elements but may include other elements not expressly listed or inherent to such process, article, or apparatus.

Further, unless expressly stated to the contrary, “or” refers to an inclusive or and not to an exclusive or. Also, any examples or illustrations given herein are not to be regarded in any way as restrictions on, limits to, or express definitions of any term or terms with which they are utilized. Instead, these examples or illustrations are to be regarded as being described with respect to one particular embodiment and as being illustrative only.

Those of ordinary skill in the art will appreciate that any term or terms with which these examples or drawings are utilized will encompass other embodiments which may or may not be given therewith or elsewhere in the specification and all such embodiments are intended to be included within the scope of that term or terms. Language designating such non-limiting examples and illustrations includes, but is not limited to: “for example,” “for instance,” “e.g.,” and “in one embodiment.”

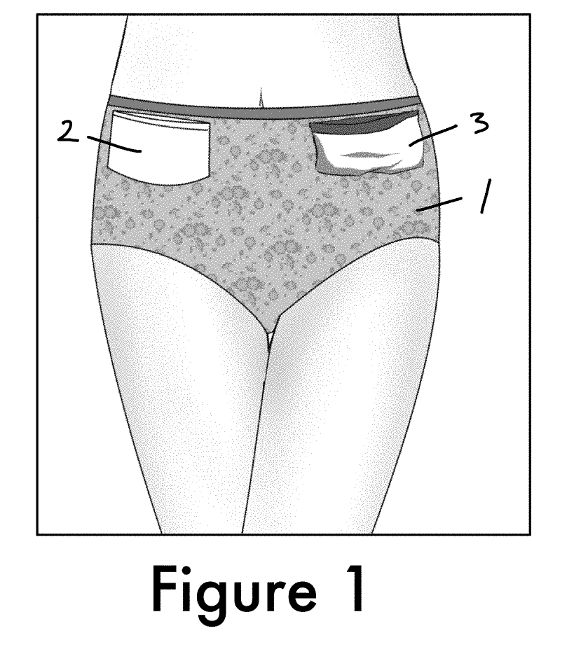

FIG. 1 illustrates an underwear article 1, along with the embodiments of the present invention. The body portion 1 may be constructed from a variety of appropriate materials and fabrics.

A non-water resistant and non-detachable pocket article 2 is attached to the body portion 1 of the underwear 1. In the embodiment, the pocket is substantially rectangular, with a greater width than height. The pocket 2 has an opening across its top. The sides and bottom of the pocket 102 are attached to the front of the body portion 1. A pocket sealing top portion seals the opening in an easily opened and reclosed fashion such that materials placed within the pocket 2 are restricted from falling out of the pocket when the pocket 2 has been sealed. In some embodiments, the pocket sealing portion may be fasteners or zippers or hooks. The opening of the pocket 2 is adjacent to and substantially parallel to the waistband defined by the upper boundary of the body portion 1. In some embodiments, the pocket 2 will utilize a front piece to construct the pocket while utilizing the front of the main body portion 1 as the back surface of the pocket.

The use of the concealed and secured pocket 2 according to embodiments of this invention allows the wearer to store supplies such as, portable catheters, wipes, pads/liners and disposable underwear, into a secure pocket of the worn clothing while maintaining the wearer's privacy. Also, this advantage is realized without any telltale signs showing through clothing, which may alert others of the wearer's medical impairments. In addition, stored supplies can be removed from the pocket 2 discreetly by the wearer reaching just inside the waistband to remove an item from the undergarment within either pocket 2.

FIG. 1 also illustrates an undergarment 1 with a detachable water resistance pocket article 3 according to the embodiment of the present invention.

The undergarment with the water resistance pocket 3 allows the wearer to privately and securely replace any soiled underwear, with a clean underwear, obtained from the detachable water resistant pocket 3, and store the soiled underwear in the water resistance pocket 3, without anybody realizing what happened and without any embarrassing mishaps and/or moments.

A detachable water resistant pocket 3 is adapted to be attached to, and removed from, the body portion 1 of the undergarment 1. The detachable water resistant pocket 3 may be defined by a front and rear panel, which are continuous, or sewn together, along a bottom edge and along the two side edges. The detachable water resistant pocket 3 has an opening approximately along its top edge. The opening may be sealed with a pocket sealing portion. In some embodiments, the pocket sealing portion may be a line of zipper, waist band, plastic slider seal, or hook.

The detachable water resistant pocket 3 is adapted to be attached to the main body 1 of the undergarment 1 along the front of the main body 1 at or just below the upper boundary of the main body 1, as seen in FIG. 1. The upper edge of the detachable water resistant pocket 3 is substantially parallel to the upper boundary of the main body 1. An attachment portion is adapted for the attachment of the detachable water resistant pocket 3 to the undergarment 1. In some embodiments, the attachment portion will be a line of zipper, waist band, plastic slider seal, or hook.

As evident from the above description, a wide variety of embodiments may be configured from the description given herein and additional advantages and modifications will readily occur to those skilled in the art. The invention in its broader aspects is, therefore, not limited to the specific details, representative apparatus and illustrative examples shown and described. Accordingly, departures from such details may be made without departing from the spirit or scope of the applicant's general invention.

What is claimed is:

1. An underwear comprising:
a body portion to be worn about the trunk predominantly below the waist of the wearer; said body portion having an elastic material defining its waistband;
two pockets, one non-detachable and the other detachable and water resistant, attached to said body portion;
the non-detachable of said pockets comprising;
a front panel as its front boundary, and
said body portion as its rear boundary,
wherein said front panel is attached to said body portion along two sides of said front panel and along the bottom of said front panel, and wherein said front panel is attached to the front of said body portion, said front panel sides extend upwards adjacent to the waistband of said body portion; and
a pocket sealing portion, said pocket sealing portion adapted to seal the top of said pocket.
the second detachable pocket comprising:
a detachable secured water resistant pocket;
the detachable water resistant pocket is adapted to be attached to, and removed from, the front panel of its front boundary. The detachable water resistant pocket may be defined by a front and rear panel, which are continuous, or sewn together, along a bottom edge and along the two side edges; and
a pocket sealing portion, said pocket sealing portion adapted to seal the top of said pocket.

2. The undergarment of claim 1 wherein said pockets are adapted to securely and discreetly hold medical and personal supplies such as, portable catheters, wipes, pads/liners and disposable underwear.
Obviously, numerous variations and modifications can be made without departing from the spirit of the present invention. Therefore, it should be clearly understood that the forms of the present invention described above, and shown in the figure of the accompanying drawing, are illustrative only and are not intended to limit the scope of the present invention.
